(19) United States
(12) Patent Application Publication
(10) Pub. No.: US 2011/0313419 A1
(43) Pub. Date: Dec. 22, 2011

(54) Hexapod External Fixation System with Collapsing Connectors

(75) Inventor: Michael W. Mullaney, Kinnelon, NJ (US)

(73) Assignee: ExtraOrtho, Inc., Memphis, TN (US)

(21) Appl. No.: 13/166,629

(22) Filed: Jun. 22, 2011

Related U.S. Application Data
(60) Provisional application No. 61/357,417, filed on Jun. 22, 2010.

(51) Int. Cl. A61F 5/04

(52) U.S. Cl. 606/56; 606/54

(57) ABSTRACT

An external fixation system includes a first base member configured to attach to a first bone segment and a second base member configured to attach to a second bone segment. A plurality of single DOF controlling elements extend between the first and second base members. The single DOF controlling elements include a crank arm having a strut interface and a base member interface, the crank arm having a first longitudinal axis and being pivotably disposed relative to one of the first and second base members. The single DOF controlling elements also include a strut extending from the strut interface toward one of the first and second base members. The strut has a second longitudinal axis, and the crank arm and strut are structurally configured so that the first and second longitudinal axes form a non-zero angle.

### PRIORITY INFORMATION

This application claims priority to U.S. Provisional Patent Application No. 61/357,417, filed Jun. 22, 2010, which is incorporated herein in its entirety.

### FIELD OF THE INVENTION

This disclosure is directed to an external fixation system for treating skeletal deformities, and more particularly, to an external fixation system having connector assemblies that extend between opposed based members and that are adjustable to treat skeletal deformities.

### BACKGROUND

Existing hexapod external fixation devices are comprised of two rings, each of which serves as a frame to attach bone fixation elements such as pins and or wires. These bone fixation elements provide a rigid connection of the frame to bone segments that are in need of manipulation in order to correct the skeletal deformity. The rings themselves are connected to and manipulated by six telescopic struts. Connections between the rings and struts necessitate the use of a spherical joint usually in the form of a ball and socket or a cardan type universal joint. To reposition one ring relative to the other, and in so doing reposition one bone element relative to the other, the lengths of the struts are adjusted by the threading of one or more elements into another. Having six struts connected in such an arrangement allows for the repositioning in all six degrees of freedom, hence the term spatial frame is commonly used to describe such devices.

Current limitations to hexapod fixation devices that utilize telescopic struts are that the minimum distance between the rings can be no less than a major fraction of the struts' shortest length and the maximum distance between the rings can be at most a fraction of the struts' longest length. Some designs seek to minimize this limitation by utilizing a nested strut approach similar to what is described in U.S. Pat. No. 5,035,094 but such an approach makes it difficult to decouple the threaded elements to allow for the rapid adjustment of the strut length as is necessary in acute manipulations. Another way of addressing this problem has been to offer struts of varying sizes. While this addresses the limitations of strut lengths, it often necessitates the changing out of strut assemblies during the course of correction which can be rather inconvenient for both the patient and the surgeon.

What is desired is a fixation system with connector assemblies that can provide a larger working envelope such that at a minimum the rings can be placed essentially coincident with one another while at a maximum the rings can be placed at such an orientation as to encompass even the most severe deformity cases. The systems and methods disclosed herein address one or more of the limitations in the prior art.

### SUMMARY OF THE INVENTION

In one exemplary aspect, the present disclosure is directed to an external fixation system. It includes a first base member configured to attach to a first bone segment and a second base member configured to attach to a second bone segment. It also includes a plurality of single DOF controlling elements extending between the first and second base members. The single DOF controlling elements include a crank arm having a strut interface and a base member interface. The crank arm has a first longitudinal axis and is pivotably disposed relative to one of the first and second base members. A strut extends from the strut interface toward one of the first and second base members. The strut has a second longitudinal axis. The crank arm and strut are structurally configured so that the first and second longitudinal axes form a non-zero angle.

In one aspect, the fixation system also includes a crank assembly that includes the crank arm and a gear reduction system. In one aspect, the gear reduction system is separable from the crank arm.

In another exemplary aspect, the present disclosure is directed to an external fixation system that includes a first ring element having perimeter edges and being configured to attach to a first bone segment and a second ring element having perimeter edges and being configured to attach to a second bone segment. The edges of the first and second ring elements define a boundary space directly therebetween. A plurality of single DOF controlling elements connect the first and second base members, and are disposed to extend at least partially outside the boundary space directly between the first and second rings.

In one aspect, the single DOF controlling elements include a crank arm and a strut structurally configured so that the first and second longitudinal axes form a non-zero angle.

In another exemplary aspect, the present disclosure is directed to a DOF controlling element for varying the relative position of two bone attachment members. The DOF controlling element includes a crank arm having a strut interface and a base member interface. The crank arm includes a first longitudinal axis and is pivotably disposed relative to one of the bone attachment members. A strut extends from the strut interface toward one of the first and second base members. The strut has a second longitudinal axis, and the crank arm and strut are structurally configured so that the first and second longitudinal axes form a non-zero angle.

In another exemplary aspect, the present disclosure is directed to a DOF controlling element for varying the relative position of first and second bone attachment members. It includes a crank assembly including a crank arm having a distal end and a proximal end, with the crank assembly being connectable to the first bone attachment member and being configured to permit motion of the crank arm about a single axis extending transverse to the crank arm. A strut has a first end and a second end, with the first end being connectable to the distal end of the crank arm. The strut's second end is connectable to the second bone attachment member.

In another exemplary aspect, the present disclosure is directed to an external fixation system that includes a first base member configured to attach to a first bone segment, the first base member having a ring-shape, includes a second base member configured to attach to a second bone segment, the second base member having a ring-shape, and includes six single DOF controlling elements disposed at substantially equal intervals extending between the first and second base members. Each of the single DOF controlling elements includes a crank arm having a strut interface and a base member interface. The crank arm has a first longitudinal axis and is pivotably disposed relative to one of the first and second base members. Each also includes a strut extending from the strut interface toward one of the first and second base members. The strut has a second longitudinal axis. The crank arm and strut are structurally configured so that the first and second longitudinal axes form a non-zero angle.

In another exemplary aspect, the present disclosure is directed to a dial indicator system for adjusting the relative position of two bone attachment members of an external fixation device. The system includes an input knob portion configured to receive an input from user and a first gear portion rotatable with the input knob portion. The gear portion includes gear teeth extending only partially around the gear portion. A second gear portion is engageable with the first gear portion, and is configured to turn only when teeth of the second gear portion engage teeth of the first gear portion. An indicator is associated with the second gear portion for viewing by the user.

### BRIEF DESCRIPTION OF THE DRAWINGS

A more complete understanding of the present disclosure and advantages thereof may be acquired by referring to the following description taken in conjunction with the accompanying figures.

FIG. 1 is an illustration of an external fixation system connected to bone segments for manipulation in accordance with one exemplary aspect of the present disclosure.

FIG. 2 is an illustration of a side view of the external fixation system of FIG. 1.

FIG. 3 is an illustration of a bottom view of the external fixation system of FIG. 1.

FIG. 4 is an illustration of a connector assembly of the external fixation system of FIG. 1 in accordance with one exemplary aspect of the present disclosure.

FIG. 5 is an illustration of the connector assembly of FIG. 4 from a top view.

FIG. 6 is an illustration of an exploded crank assembly of the connector assembly of FIG. 4 in accordance with one exemplary aspect of the present disclosure.

FIG. 7 is an illustration of a top view of the crank assembly of FIG. 6 in assembled form.

FIG. 8 is an illustration of a cross-sectional view of the crank assembly of FIG. 4 taken through lines 8-8 in FIG. 4.

FIG. 9 is an illustration of a cross-sectional view of the crank assembly of FIG. 4 taken through lines 9-9 in FIG. 7.

FIG. 10 is an illustration of a cross-sectional view of the crank assembly of FIG. 4 taken through lines 10-10 in FIG. 7.

FIG. 11 is an illustration of the crank assembly of FIG. 4 without the housing or input and indicator systems, showing an adjustment mechanism and drive shaft.

FIG. 12 is an illustration of a cross-sectional view of the crank assembly of FIG. 4 taken through lines 12-12 in FIG. 7.

FIG. 13 is an illustration of a knob ring assembly of the crank assembly of FIG. 4 in accordance with one exemplary aspect of the present disclosure.

FIG. 14 is an illustration of a pinion gear of the crank assembly of FIG. 4 in accordance with one exemplary aspect of the present disclosure.

FIGS. 15-17 are illustrations showing incremental assembly of an input and indicator system in accordance with one exemplary aspect of the present disclosure.

FIGS. 18-21 are illustrations showing the bone fixation device of FIG. 1 in different configurations.

FIGS. 22-25 are illustrations of an input and indicator system 135 in accordance with a different exemplary aspect of the present disclosure that may be implementable on the external fixation system in FIG. 1.

FIG. 26 is an illustration of a side view of another external fixation system in accordance with one exemplary aspect of the present disclosure.

FIG. 27 is an illustration of a top view of the external fixation system of FIG. 26.

FIG. 28 is an illustration of a crank assembly of the external fixation system of FIG. 26 in accordance with one exemplary aspect of the present disclosure.

FIG. 29 is an illustration of an exploded view of the crank assembly of FIG. 28.

FIG. 30 is an illustration of a cross-sectional view of the crank assembly of FIG. 28.

### DETAILED DESCRIPTION

Reference is now made in detail to exemplary embodiments of the invention, examples of which are illustrated in the accompanying drawings. Wherever possible, the same reference numbers are used throughout the drawings to refer to the same or like parts.

The present disclosure is directed to an exemplary bone fixation device having a larger working range than prior bone fixation devices. The fixation device connects to bone segments and, through incremental adjustments made to the fixation device over time, can be used to treat and correct bone deformities. Base members connect to the bone fragments, and connector assemblies separate the base members. The connector assemblies include a joint disposed between their ends that enables the connector assemblies to collapse so that the base members can be near each other, and also enables the connector assemblies to move to a substantially linear condition, creating a maximum separation distance of the base members. This provides a greater range of travel distance for the base members than could be achieved in prior devices that use struts that extend only linearly between base members. The connector assemblies can, at a minimum, orient the base members essentially coincident with one another while, at a maximum, the base members can be placed at such an orientation as to encompass even the most severe deformity cases.

In addition, to enable incremental adjustment for deformity correction over long periods of time, the fixation device herein includes an indicator system that displays to a user the setting in units of the particular connector assembly. A surgical treatment plan may include instructions to adjust one or more connector assemblies to achieve a desired result. These instructions may include incrementally advancing or retracting the connector assemblies a specific amount consistent with the indicator system.

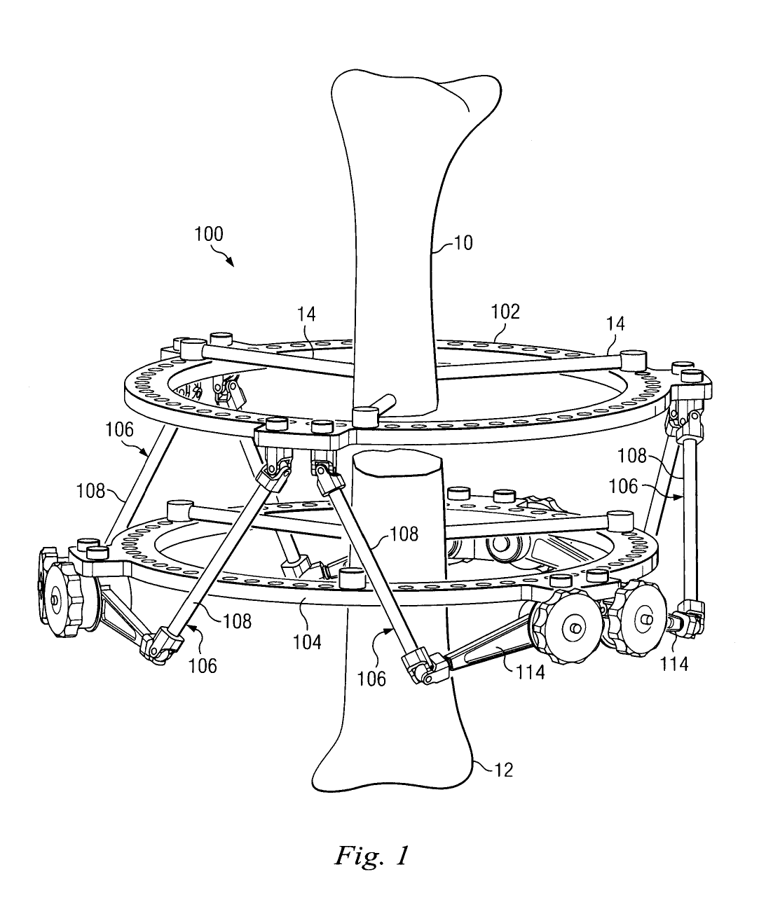

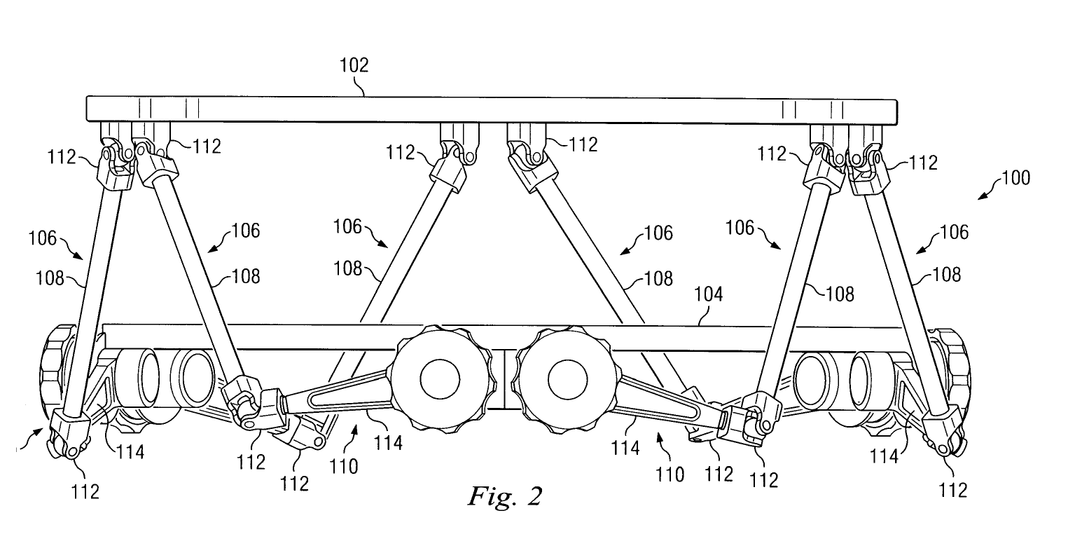

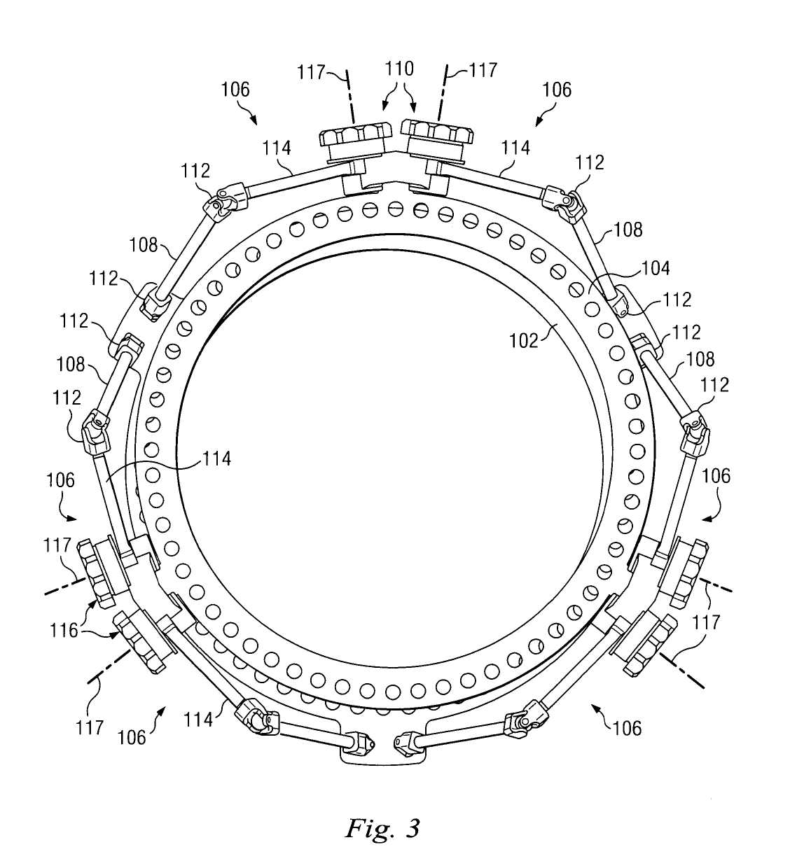

FIGS. 1-3 depict an exemplary bone fixation device 100. In this example, the bone fixation device is a hexapod. This embodiment of the bone fixation device 100 includes a first base member as a first ring element 102 and a second base member as a second ring element 104, connected by six degree-of-freedom controlling elements, referred to herein as connector assemblies 106 that control the relative positioning of the ring elements 102, 104. That is, one by one as they are added to fixation device 100, they remove a single degree of freedom from the mobility of the first ring element 102 relative to the second ring element 104. As such, the connector assemblies cooperate to orient the first element 102 relative to the second ring element 104. FIG. 1 shows the fixation device 100 connected to first and second bone segments 10, 12. FIGS. 2 and 3 show the bone fixation device 100 without the bone segments from the side and bottom respectively. Referring to FIG. 1, the fixation device 100 connects to the first and second bone segments 10, 12 using bone pins 14. The bone pins 14 are rigidly connected to the first or second ring elements 102, 104 to provide stabilization support to the bone segments during the deformity correction procedure or during the desired period of fixation. In this example, the bone pins 14 are connected to and extend from the ring elements 102, 104 by adjustable joints or pivoting heads that may be tightened in place to secure the pins 14 in place relative to the fixation device 100. Since the device 100 may be used to treat deformities, instead of being used only for stabilization during healing of fractures for example, the fixation device 100 is configured to be adjusted over the course of treatment, which depending on the deformity and the amount of correction, can take up to or over a year. Over this time, the bone fixation device 100 is incrementally adjusted to comply with a treatment plan and gradually correct the deformity.

As can be seen in FIGS. 1-3, the connector assemblies 106 extend from and connect to the first and second ring elements 102, 104. In this example, as can be seen in FIG. 3, the connector assemblies 106 extend outside the ring elements 102, 104. That is, if the ring elements define a boundary, the connector assemblies 106 extend outside the boundaries. In some examples, as can the first and second ring elements 102, 104 comprise edges, and the connector assemblies 106 are disposed tangent to the edges, and travel in a plane tangent to the edges.

As will become apparent from the following description, the connector assemblies 106 may be incrementally adjusted to manipulate the relative positioning of the ring elements 102, 104. As is desirable for deformity correction, the adjustments may be made in fine, controlled increments, providing accuracy for controlled, methodical treatments.

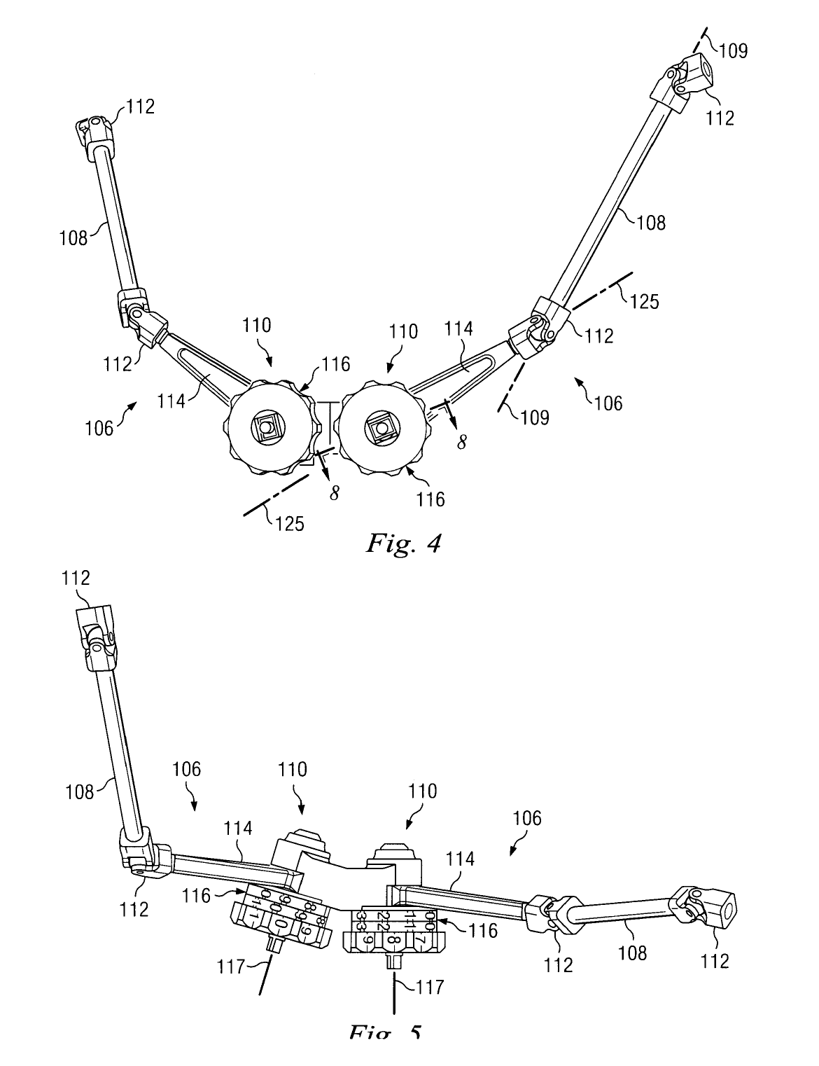

FIGS. 4 and 5 show the connector assemblies 106 independent of the ring elements 102, 104. Referring to these figures, the connector assemblies 106 each include a fixed length strut 108 and a crank assembly 110. The fixed length strut 108 includes a longitudinal axis 109. Spherical joints 112 are disposed at each end of the fixed length strut 108 and connect the fixed length strut 108 to the first ring element 102 and connect the fixed length strut 108 to the crank assembly 110. In this example, the spherical joints 112 are shown as universal joints capable of providing relative rotation about a single point. In the example shown, the spherical joint 112 is a part of a structure that fits over adjacent ends of the fixed length strut 108 and the crank assembly 110 to connect them. In other examples, elements of the spherical joint 112 may be integral with one or both of the crank arm 114 and the fixed length strut 108. The fixed length strut 108 is merely a rigid rod extending from one end to the other.

The crank assembly 110 connects the fixed length strut 108 to the second ring element 104. It includes a crank arm 114 and an adjustment assembly 116. While the first ring element 102 connects to the connector assembly 106 at a spherical joint 112 providing multiple degrees of freedom, the second ring element 104 connects to the crank assembly 110 in a manner that provides only pivoting movement about an axis of revolution 117. In some examples, the axis of revolution 117 itself revolves around a parallel axis at some degree of eccentricity.

Because it pivots about the axis of revolution 117, the spherical joint connecting the crank arm 114 and the fixed length strut 108 travels in an arc in a single plane. The fixation device 100 may be adjusted by effecting a rotation of the crank arm 114 about the axis of revolution 117 thereby changing the location of the spherical joint 112 attached to it. The advantage of such an arrangement is that the connector assemblies 106 can be made to fold back onto themselves allowing for a much greater range of motion than can be obtained using a linearly adjustable length strut.

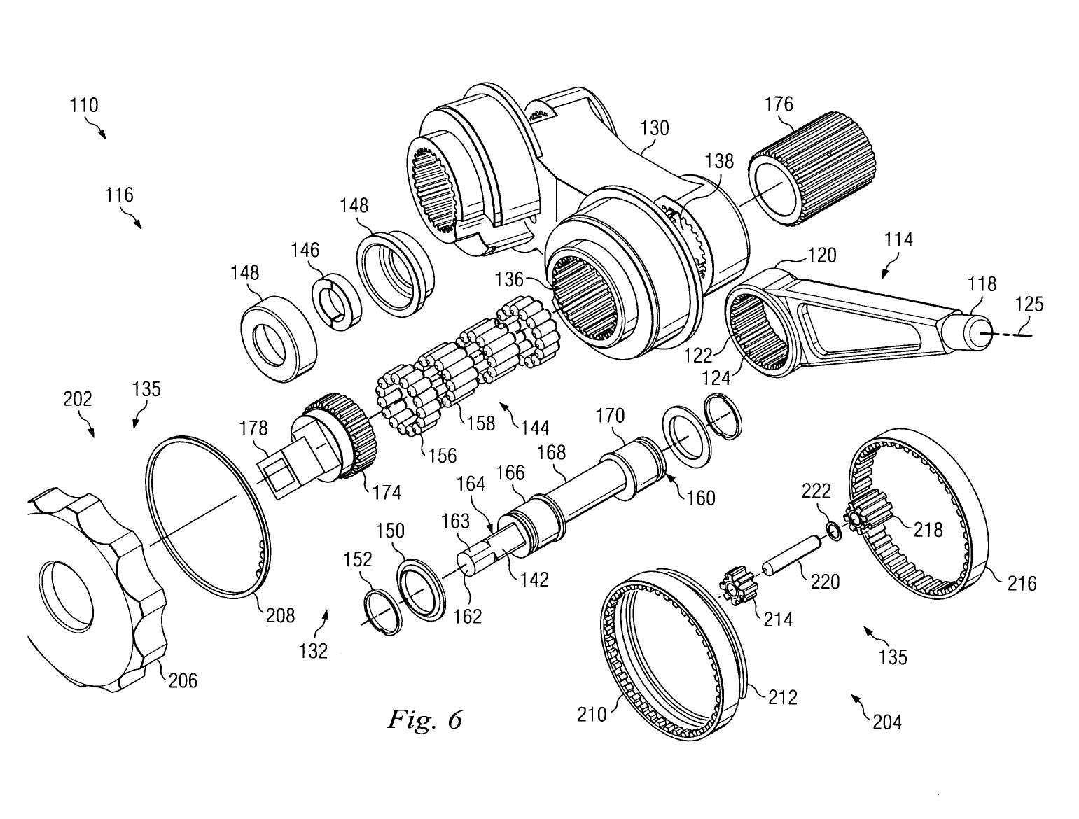

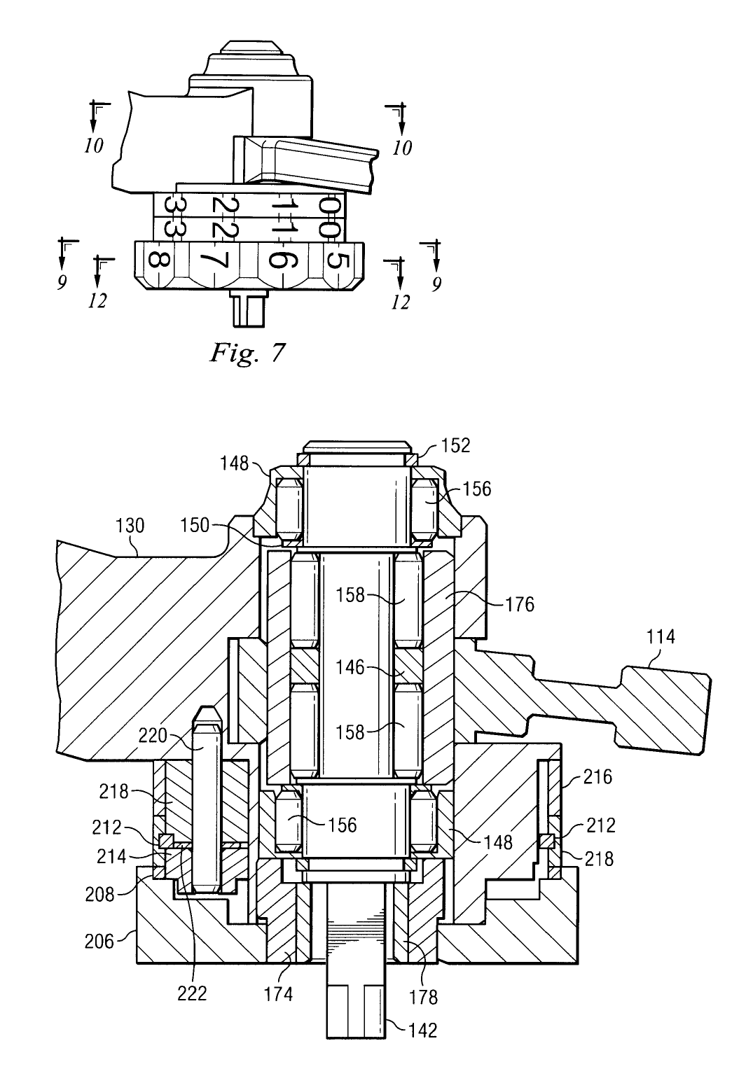

FIG. 6 shows the crank assembly 110 in exploded form. FIG. 7 shows a side view of the crank assembly 110. The crank arm 114 extends between the adjustment assembly 116 and the fixed length strut 108. It includes a distal end 118 that connects to the fixed length strut 108 via the spherical joint 112 (not shown in FIG. 6) and includes a proximal end 120 that connects with the adjustment assembly 116. In this example, the proximal end 120 of the crank arm 114 includes a through passage 122 with gear teeth 124 formed thereon for engaging gear teeth of the adjustment assembly 116, as explained below. A longitudinal axis 125 extends through the distal and proximal ends. This axis 125 may be disposed at a non-zero angle relative to the axis 109 of the fixed length adjustable strut 108 as shown in FIG. 4.

The adjustment assembly 116 cooperates with and drives the movement of the crank arm 114 about the axis of revolution 117. The adjustment assembly 116 includes a housing 130, a drive assembly 132, an adjustment mechanism 134, and an input and indicator system 135. FIG. 8 shows a cross-section of the adjustment assembly 116 taken through the lines 8-8 in FIG. 4.

Referring to both FIGS. 6 and 8, the housing 130 is configured to attach securely to the second ring element 104 and limit any relative movement of the location of the crank assembly 110. Here, the housing 130 includes an internal bore 136 and a lateral slot 138. The internal bore 136 is formed as a ring gear with a series of gear teeth engagable with gears in the adjustment mechanism 134. In one example, the housing 130 has thirty gear teeth machined therein. The lateral slot 138 is configured to receive the proximal end 120 of the crank arm 114. When the crank arm 114 is disposed within the lateral slot 138, the passage 122 of the crank arm and the internal bore 136 of the housing both define the axis of revolution 117, about which the crank arm pivots. As indicated above, in this example, the housing 130 is shared between two adjacent crank assemblies 110. This can facilitate connection to the ring elements 102, 104. However, in other embodiments, each crank assembly has a separate and independent housing. In such embodiments, each housing connects independently to the associated ring element with some number of crank assemblies being connected to both of the ring elements rather than all crank assemblies being connected to just one ring element.

The drive assembly 132 projects into both the internal bore 136 of the housing 130 and into the passage 122 in the crank arm 114. Referring to FIGS. 6 and 8, the drive assembly 132 includes a drive shaft 142, a bearing stack 144, a bushing 146, and two bearing caps 148. Bearing spacers 150 and retaining rings 152 cooperate with the bearing stack 144 to maintain the bearing stack 144 in place.

The bearing stack 144 includes two arrays of long needle bearings 154 and two arrays of short needle bearings 156. The arrays of long needle bearings 154 are separated from each other by the bushing 146, as shown in FIG. 8. The bearing spacers 150 and the retaining rings 152 separate the long needle bearing arrays from the short needle bearing arrays. The bearing caps 148 support the short needle bearing arrays 156 and maintain them in position on the drive shaft 142. The bearing arrays 154, 156 act against and provide bearing support for rotation of the drive shaft 142 about the axis of revolution 117.

The drive shaft 142 includes a distal end 160 and a proximal end 162. Starting at the proximal end 162, the drive shaft 142 includes an extension region 164 having a square boss 163. It also includes a first journal section 166, a second eccentric journal section 168, and a third journal section 170. The first and third journal sections 166, 170 are carried by the short needle arrays 156. The eccentric second journal section 168 between the first and third journal sections 166, 170 interfaces with the long needle arrays 156.

The adjustment mechanism 134 includes the components that effectively carry out the controlled rotation of the crank arm 114 relative to the adjustment assembly 116. The adjustment mechanism 134 includes an input stage gear 174, an output stage gear 176, and an Oldham coupler 178.

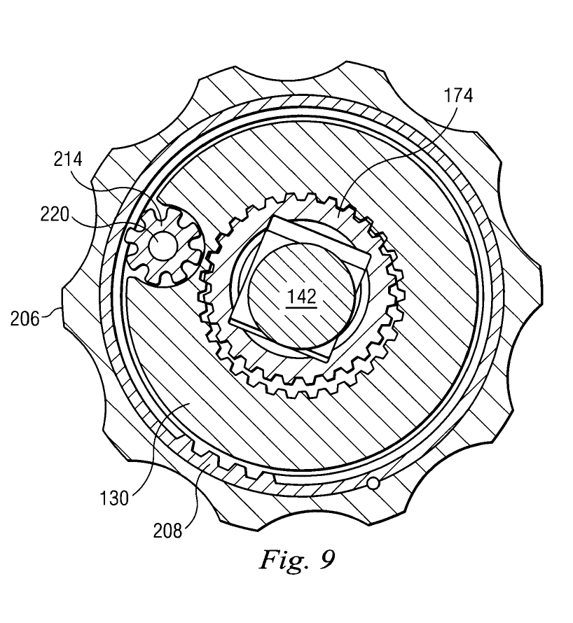

The input stage gear 174 is a pinion gear disposed to extend over the extension region 164 of the drive shaft. It fits within the internal bore 136 of the housing 130 and has outwardly facing teeth arranged to engage the ring gear teeth on the internal bore 136. The cross section in FIG. 9, taken though lines 9-9 in FIG. 7, shows the input stage gear 174 eccentrically engaged with the ring gear teeth on the internal bore 136 of the housing 130. The input stage gear 174 includes a different number of teeth than the number of teeth of the internal bore 136 of the housing 130. In this example the input stage gear 174 includes twenty-eight teeth and the internal bore 136 includes thirty teeth. As explained further below, this differential creates a first gear reduction system.

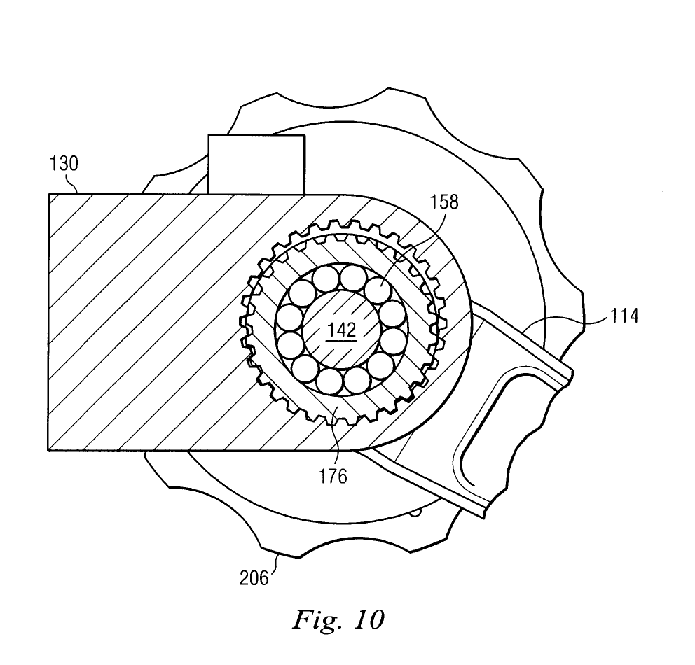

The output stage gear 176 is carried by the long needle arrays 154 that are disposed about the eccentric second journal section 168 of the drive shaft 142. It is a pinion gear with teeth arranged to engage the ring gear teeth on the internal bore 136 of the housing 130. The output stage gear 176 also extends through the passage 122 in the crank arm 114. The cross section in FIG. 10, taken though lines 10-10 in FIG. 7 shows the output stage gear 176 eccentrically engaged with the ring gear teeth on the internal bore 136 of the housing 130. Like the input stage gear 174, the output stage gear 176 includes twenty-eight teeth and, as indicated above, the internal bore 136 includes thirty teeth. As explained further below, this differential creates a second gear reduction system.

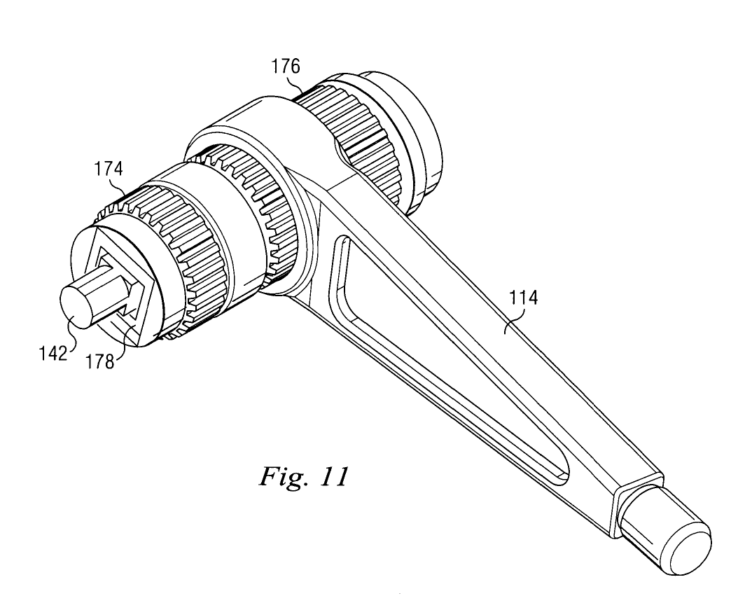

The outer teeth of the output stage gear 176 mesh with the teeth 124 on the crank arm. The output stage gear 176 and the crank arm 114 have matching profiles, so that the crank arm 114 is rotationally fixed relative to the output stage gear 176. As such, as the output stage gear 176 rotates, the crank arm 114 also rotates. For reference, FIG. 11 shows the crank arm 114, the drive shaft 142, and adjustment mechanism 134 with its input stage gear 174, output stage gear 176, and Oldham coupler 178.

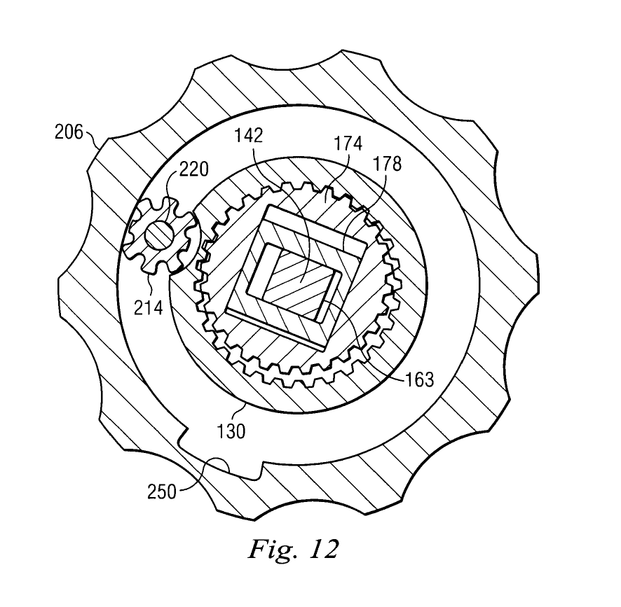

The Oldham coupler 178 couples the first gear reduction system with the input stage gear 174 to the second gear reduction system with the output stage gear 176. It has a rectangular slot 186 that mates with the square boss 163 on the drive shaft 142. In the example shown, the outside of the Oldham coupler 178 is also rectangular and is fitted within a square hole in the input stage gear 174. The cross section in FIG. 12, taken though lines 12-12 in FIG. 7, shows the square boss 163 in the Oldham coupler 178, with the Oldham coupler fitted in the input stage gear 174. Since the square boss 163 on the drive shaft 142 is the same dimension as the lesser side of the rectangular slot 186 in the Oldham coupler, sliding is permitted in one direction while relative rotation is not, as can be understood from FIG. 12. Similarly the larger dimension of the outside of Oldham coupler 178 is the same as the square bore dimension in the output stage gear 176 allowing sliding along the lesser side of the Oldham coupler 178. This relative sliding action takes place at right angles to one another allowing the eccentric input stage gear 174 to smoothly couple with the concentric drive shaft 142 while not allowing relative rotation. It should be noted that similar face to face arrangements may also be utilized whereby both the input and output members have either a tongue or a groove and a disk in the middle has a mating tongue or groove on opposite faces that are orthogonally opposed. This arrangement is commonly known as an Oldham coupling but other such couplings that allow for the coupling of eccentric shafts could also be utilized. Likewise other gear reduction mechanisms could be used as well.

The input and indicator system 135 receives inputs from a health care provider or the patient and visually displays the resulting amount of adjustment in reference units. This enables a health care provider or patient to perform their own adjustments according to the treatment plan. The input and indicator system 135 permits a user to easily track and easily control the adjustments made to the device 100.

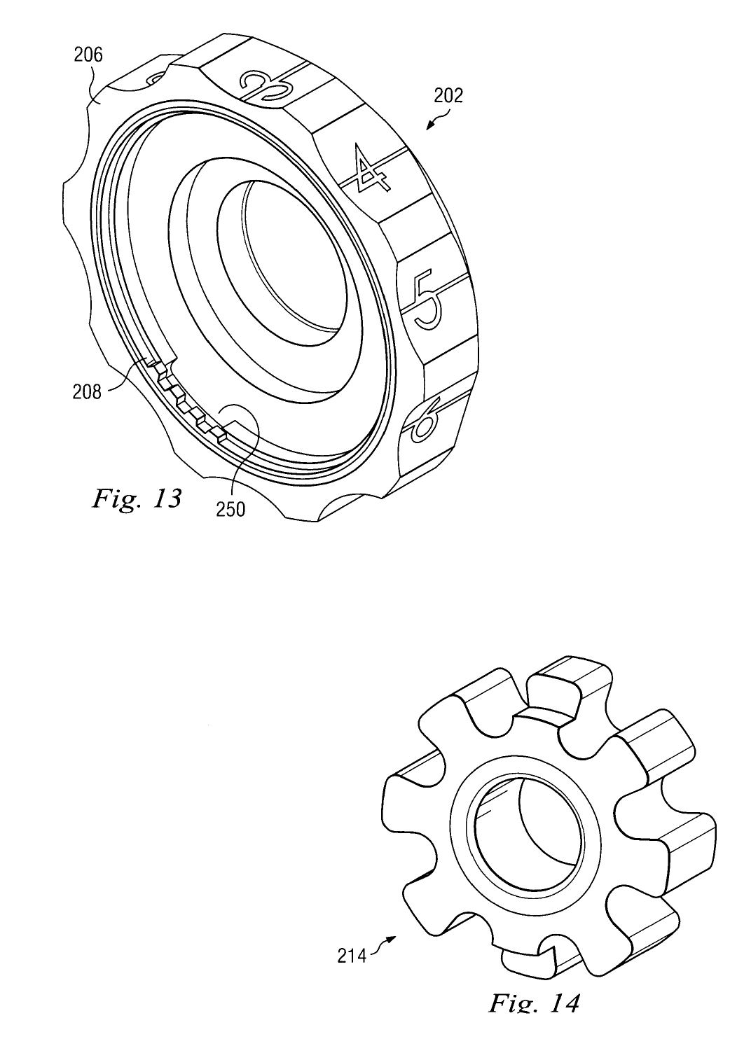

The input and indicator system 135 allows a user to manipulate the connector assembly 106 and provides a user with a readout related to the angulation of the crank arm 114. It includes a knob ring assembly 202 and a dial ring assembly 204. The knob ring assembly 202 includes an input knob 206 and a ring gear 208. While FIG. 7 shows the knob ring assembly 202 in exploded form, FIG. 13 shows the knob ring assembly 202 in assembled form. As shown in the figures, the input knob 206 includes indicators formed thereon and is shaped for gripping by a user. As such, it has a series of finger indentations for manipulation by a user. In this example, the ring gear 208 has all but four teeth machined away. Accordingly, this set of teeth extends along only a short segment of the ring. The input knob 206 connects with and drives the input stage gear 174. As such, it has a central opening though which the drive proximal end of the drive shaft 142 extends.

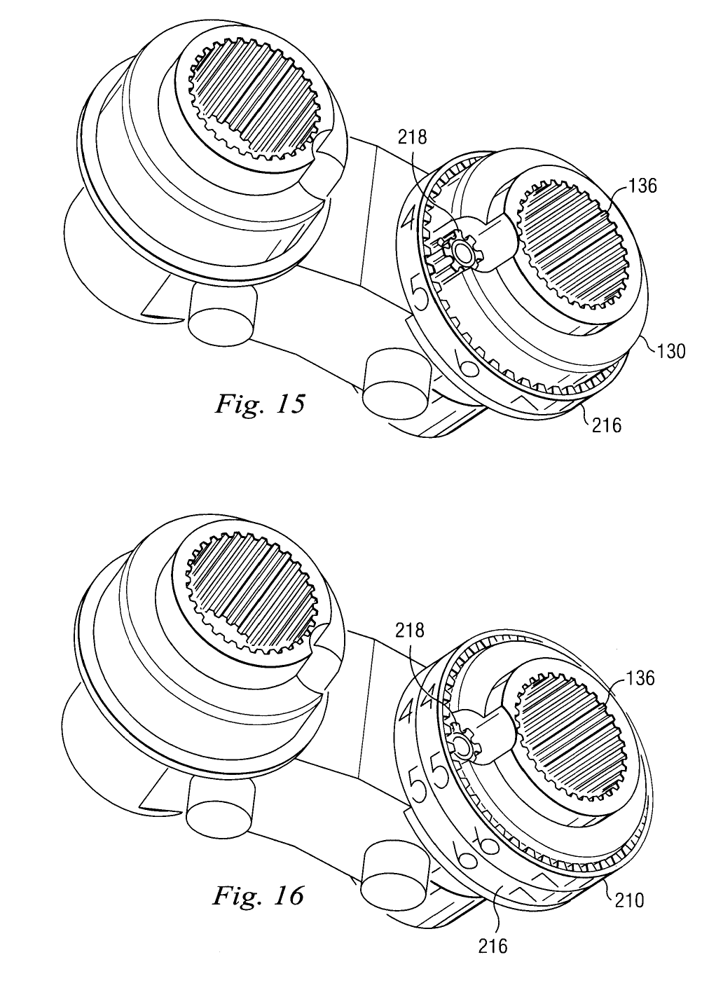

The dial ring assembly 204 includes a first dial 210, a pinion stop ring 212, a first pinion gear 214, a second dial 216, and a second pinion gear 218. A pinion shaft 220 connects the first and second pinion gears 214, 218 and a bushing 222 is disposed between the gears. As can be seen, both the first and second dials 210, 216 are ring gears and have reference indicators imprinted or formed on the outer-facing surfaces. In this example, the indicators are digits. The ring gear first dial 210 meshes with the first pinion gear 214. The first pinion gear 214 has eight teeth, with the fourth and eighth teeth being partially cut away. In addition to being shown in FIG. 6, this is also shown in greater detail in FIG. 14. The first pinion gear 214 is disposed to mesh with the ring gear 208 of the knob ring assembly 202 where the fourth and eighth teeth are cut away. This can be seen in the cross-section of FIG. 12. It also meshes with the ring gear of the first dial 210 where the fourth and eighth teeth are not cut away. The second pinion gear 218 is configured in a manner similar to the first pinion gear 214, having eight teeth with a portion of the fourth and eighth teeth cut away. The second pinion gear 218 interfaces with the pinion stop ring 212 with the portion of the gear having cut away teeth and interfaces with the ring gear of the dial 216 with the portion of the gear not having teeth cut away. As will become clear from the discussion further below, the input knob 206 and the first and second dials 210, 216 cooperate to provide the user with a readout related to the angulation of the crank arm 114. With the input knob 206 providing a first digit, the first dial 210 providing a second digit, and the second dial 216 providing the third digit, the input and indicator system 135 has a ones place, a tens place and a hundreds place for a total range of 0 to 999. This is shown, for example, in FIG. 7. Accordingly, a user can turn the input knob 206 to rotate the crank arm 114 and the input and indicator system 135 shows the reference units. Accordingly, the treatment plan may include manipulating the hexapod by adjusting specific connector assemblies 106 by a specific number of units over a specific length of the treatment. For example, the treatment plan may prescribe a weekly input to rotate the knob 206 five clicks or units from its current setting to a new setting.

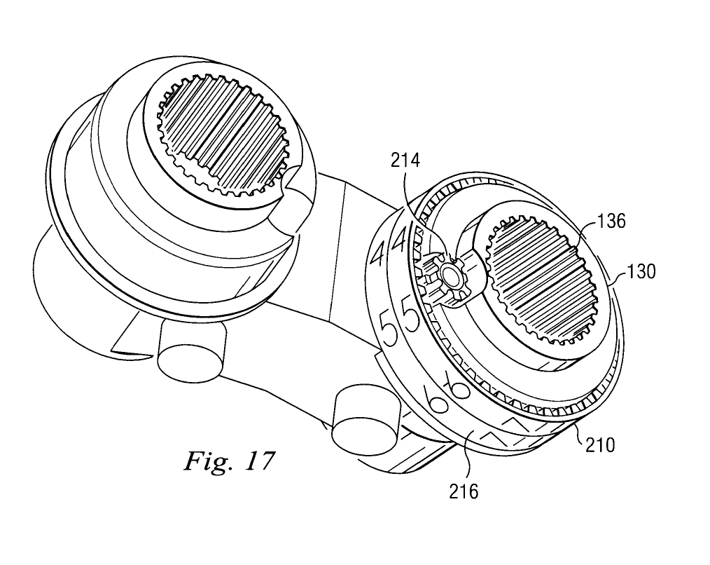

FIGS. 15-17 show a partial assembly of the dial ring assembly 204. Referring first to Fig, 15, the second dial 216 fits about a portion of the housing 130. The second pinion gear 218 fits within a gear slot formed in the housing 130. As can be seen, the second pinion gear 218 engages the ring gear of the second dial 216 with all eight teeth, but also projects above the second ring gear. FIG. 16 shows the addition of the first dial 210 and the pinion stop ring 212. The first dial 210 and pinion stop ring 212 engage only the portion of the second pinion gear 218 having every fourth tooth cut away. It cooperates in the manner described above with reference to the input knob 206 and ring gear 208 having only four teeth. FIG. 17 shows the addition of the first pinion gear 214. The first pinion gear engages the first dial 210 with the portion having all the teeth, but projects above the first dial 210 and is configured to engage the knob ring assembly 202 with the input knob 206 and the ring gear 208 as described above.

In use, a surgeon may insert bone pins 14 into bone segments, and attach the bone pins 14 to the first and second ring elements 102, 104. Using methods known in the art, the position of the bone segments may be characterized in their current location and in a desired, corrected location. A treatment plan may then be developed that includes incremental adjustments over time to move the bone fixation device 100 from the current position on the bone segments to a final position where the bone segments are in a desired corrected location. For example, the treatment plan may include specific instructions to be carried out for each of the connector assemblies 106. That is, the instructions to achieve the desired final position may be different for each of the connector assemblies 106, although the ultimate goal is to have the connector assemblies cooperate to arrive at the position that best corrects the deformity.

With the fixation device 100 connected to opposing bone segments and with the treatment plan created, a user may begin incrementally adjusting the connector assemblies according to the plan to achieve the desired result. For example, a user may adjust one or more the connector assemblies at specific intervals, such as for example, once a week, to achieve desired manipulation of the bone segments. To adjust a connector assembly 106, the user rotates the input knob 206. For example, the user may be instructed according to the treatment plan to move the input knob to advance the units on the knob and dials a certain amount of digits. An example would be to move the dial three digits, from 298 to 301.

To do this, the user grasps and rotates the input knob 206. The input knob 206 is connected with and fixed relative to the input stage gear 174. The following description will first describe how the input and indicator system 135 responds to the rotation, and then will describe how the crank assembly 110 as a whole responds to move the crank arm 114 to displace the fixed length strut 108 and effect relative movement of the first and second ring elements 102, 104.

As indicated above, the input knob 206 shows increments 0 through 9 and serves as the ones place in the reference units. The input knob 206 rotates the ring gear 208 that has all but four of its teeth machined away, as shown in FIG. 13. When assembled, the input knob 206 is mated with the pinion gear 214, which revolves around the pinion shaft 220. The pinion gear 214 has eight teeth with every fourth tooth being cut away, as described above. As such, it does not have the same face width as the remaining teeth. Because of the shape, every one revolution of the knob ring assembly 202, with the input knob 206 and ring gear 208, the pinion gear 214 has a four tooth revolution. When the pinion gear 214 is not being driven by the teeth on the ring gear 208, the pinion gear 214 is locked in place by the inner surface of the input knob 206, which makes contact with the undisturbed teeth adjacent to the tooth that is cut away, preventing rotation, as can be seen in the cross-section FIG. 11. A milled pocket 250 in the interior of the input knob 206 in FIGS. 11 and 12 sits below the four teeth of the ring gear 208. This pocket 250 allows the pinion gear 214 to rotate when the gear 214 engages the four teeth of the ring gear 208. Once the four teeth pass, the pinion gear 214 is once again locked in place by the inner surface of the input knob 206. This same approach is carried one stage further in the case of the dial 216 and pinion gear 218. That is, the pinion stop ring 212 is fixed relative to the dial 210 and includes four teeth that cooperate with the pinion gear 218. Accordingly, when the first dial 210 makes one full revolution, the second dial 216 will advance four gear teeth, which equates to a one-tenth revolution. The result is a dial indication system that has a ones place on the input knob 206, a tens place on the first dial 210, and a hundreds place on the second dial 216 for a total range of 000 to 999 units. As such, using the reference numbers, a user can easily see the reference numbers representing the advancement or retraction of the crank arm 114.

The following description discusses how the crank assembly 110 as a whole responds to rotation of the crank arm 114. The input knob 206 is fixed in place relative to the input stage gear 174. As such, rotation of the input knob 206 also rotates the input stage gear 174. Since manipulation of the fixation device may be done in fine, very small increments, the crank assembly 110 includes two gear reduction systems that convert motion at the input knob 206 into motion of the crank shaft 114.

The input stage gear 174 has twenty-eight teeth of the same pitch as the ring gear in the internal bore 136 of the housing 130. It is eccentric such that the teeth of the ring gear in the internal bore 136 of the housing 130 meshes with the input stage gear 174, as shown in FIG. 9. The input knob 206 drives the input stage gear 174. The input knob 206 also has an eccentric bore that mates with input stage gear 174. Rotation of the input knob 206 relative to the housing 130 causes each successive tooth of the input stage gear 174 to engage with the teeth in the internal bore 136 of the housing 130. Accordingly, one full rotation of the input knob 206 results in one full orbit of the input stage gear 174 within the internal bore 136 of the housing 130, which results in a two tooth rotation of the input stage gear 174 about its own axis. Because one rotation of the gear advances the gear only two teeth, the input stage gear 174 has a reduction ratio of 14:1.

The Oldham coupler 178 couples the input stage gear 174 to the drive shaft 142, and subsequently, to the output stage gear 176. The rectangular slot 186 in the Oldham coupler 178 mates with the square boss 163 on the drive shaft 142 and allows the drive shaft 142 to turn smoothly as it rotates with the eccentric input stage gear 174, without relative rotation of the input stage gear 174 and the drive shaft 142.

As described above and as shown in FIG. 8, the second journal section 168 of the drive shaft 142 is eccentric. The long needle bushing array 154 fits around the second journal section 168 and carries the output stage gear 176. The teeth of the output stage gear 176 are meshed with the ring gear teeth of the internal bore 136 of the housing 130. Accordingly, as the drive shaft 142 rotates, the external teeth on the output stage gear 176 engage with successive internal teeth in the internal bore 136 of the housing 130. Since the output stage gear 176 has twenty-eight teeth and the housing internal bore 136 has thirty teeth, each complete rotation of the drive shaft 142 results in a two tooth rotation of the output stage gear 176 about its own axis. Accordingly, fourteen such rotations of the drive shaft 142, will therefore result in one complete revolution of the output stage gear 176. Similar to the input stage gear 174, the output stage gear 176 has a reduction ratio of 14:1 resulting in a total reduction ratio of 196:1.

The crank arm 114 is meshed with the output stage gear 176. Accordingly, as the output stage gear 176 rotates, after having been reduced at the 196:1 ratio, the crank arm 114 also rotates at that ratio relative to the housing 130 and relative to the second ring element 104. Since the crank arm 114 connects at its distal end 118 to the fixed length strut 108, displacement of the crank arm 114 results in movement of the fixed length strut 108 relative to the second ring element 104. Since the first ring element 102 is connected to the fixed length strut 108, movement of the fixed length strut 108 results in movement of the first ring element 102 relative to the second ring element 104. This results in manipulation of the bone segments.

In the example shown and described above, the gear reduction is a two stage cycloidal with each stage having a reduction ratio of 14. It should be apparent that other gear reduction mechanisms and ratios could be used as well.

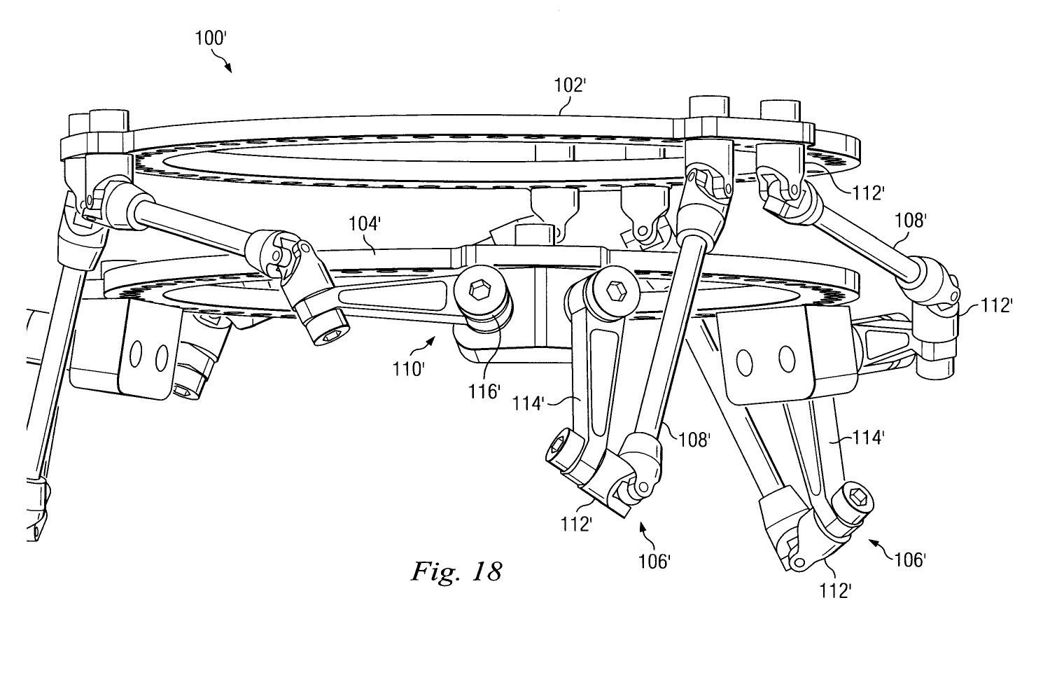

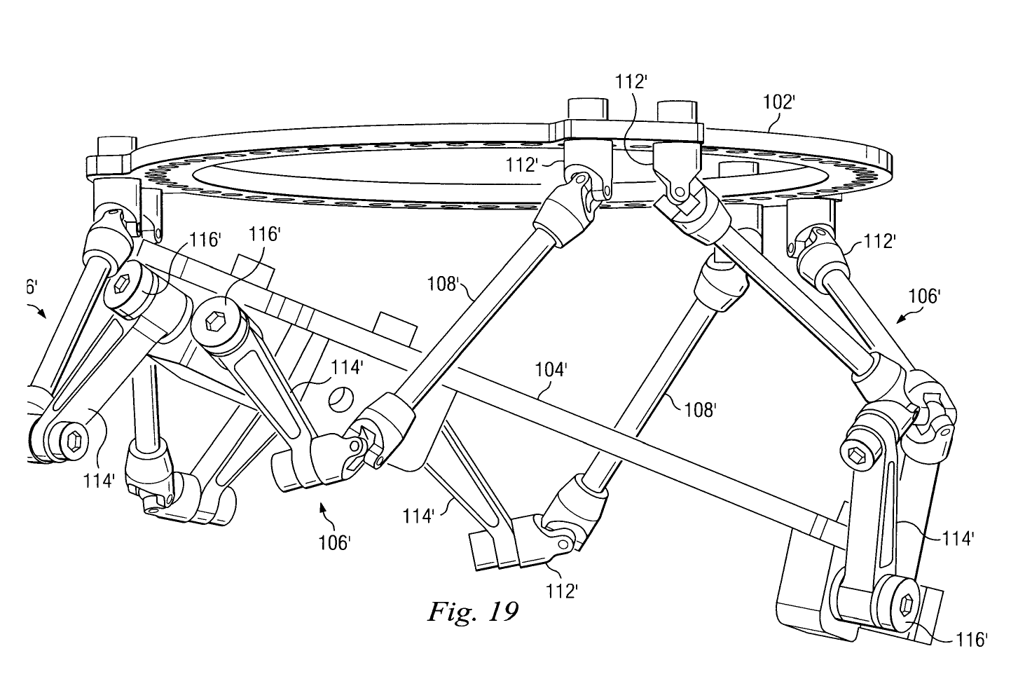

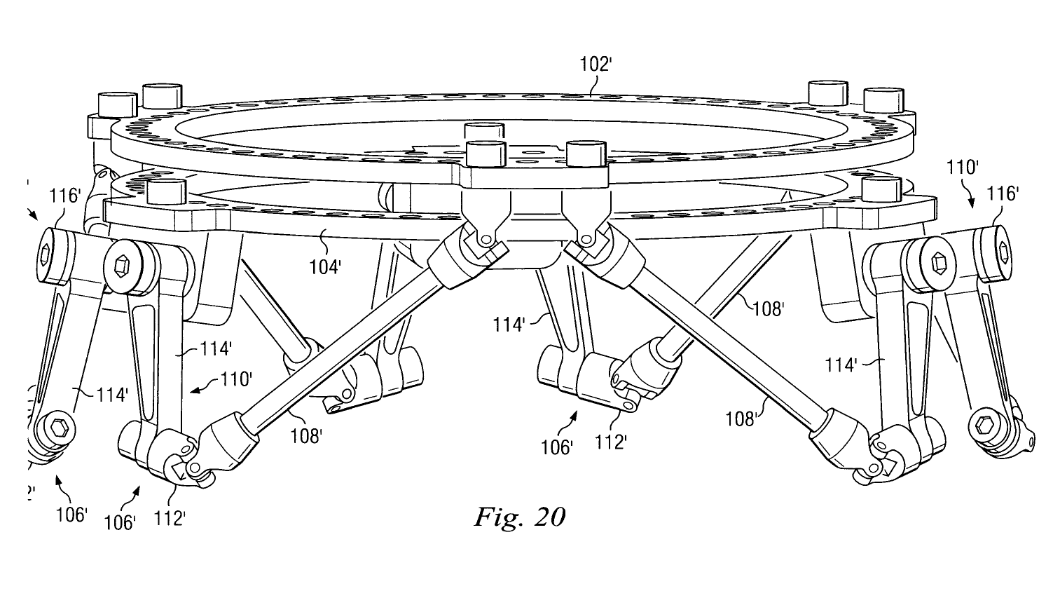

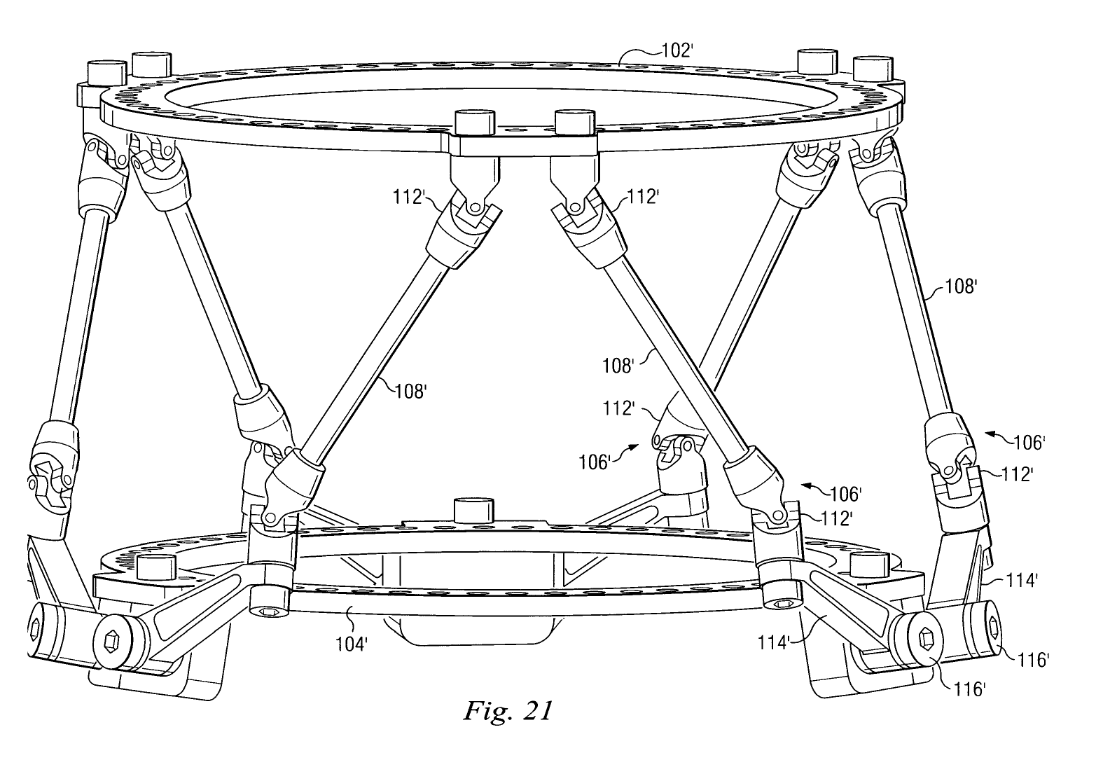

FIGS. 18-21 show a bone fixation device 100′ in different configurations. FIG. 18 shows the device in a rotated configuration, FIG. 19 shows the device in an angulated configuration, FIG. 20 shows the device in a collapsed configuration, and FIG. 21 shows the device in an extended configuration. It should be apparent that the same configurations are obtained with the bone fixation device 100 discussed above, and the bone fixation device 100′ is provided here in part to show the variety of configurations obtainable by bone fixation systems arranged with non-linear connector assemblies.

Also, as can be seen, these bone fixation devices 100′ include crank assemblies 110′ with crank arms 114′ and adjustment assemblies 116′. In this case however, the adjustment assemblies 116′ do not include the drive assemblies and adjustment mechanisms as described above. As such, the crank arm 114′ and the gear reduction system, or portions thereof, may be separable. In one example, the drive assemblies and adjustment mechanisms are disposed on a separate tool, attachable to the adjustment assemblies 116′. For example, here the adjustment assemblies 116′ include a housing and shoulder screws that cooperate with the crank arm 114 to secure the crank arm 114′ in place relative to the ring elements. The shoulder screws or other components within the adjustment assemblies 116′ may include interdigitations that mesh with interdigitations on the crank arm 114 to secure the crank arm 114′ when it is not being adjusted. The separate adjustment tool selectively connects or affixes to the adjustment assembly 116′ to rotate the crank arm 114′. In this example, therefore, the tool may comprise components of the adjustment mechanism 134 described above and/or other components of the adjustment assembly 116′. Particularly, the tool may carry a housing with gear reducing elements disposed therein.

In use, the tool is arranged to affix to the crank assembly 110′. It may affix to the crank arm 114′ and the adjustment assembly 116′ at the same time, either outside or inside the adjustment assembly 116′. Once affixed so that undesired relative motion of the crank arm 114′ will not occur, the tool may be used loosen the shoulder screw of the adjustment assembly 116′. Poker chip interdigitations within the adjustment assembly 116′ may secure the crank arm 114′ in position when the shoulder screw is tight. With the shoulder screw loosened however, the interdigitations may disengage or the tool may disengage the interdigitations, and the tool is then relied upon to fix the relative location of the crank arm 114′ so that position will not be lost. With the crank arm 114′ affixed to the tool, the tool may be used to adjust the position of the crank arm relative to the ring elements to adjust the connector assemblies 106′. To do this, the tool may receive an input from a user, and using a gear reduction like that described above, rotate the crank arm 114′. After the desired rotation of the crank arm, the tool is used to re-tighten the shoulder screw to re-fix the crank arm in position. With the crank arm secured in its new desired position the tool is removed from the crank assembly 110′.

Accordingly, in some examples, the tool provides for the rotation of the crank arm 114′ utilizing either a direct drive approach or a geared approach to lessen the input torque required to turn the crank arm and to allow for more precise adjustment. The engagement mechanism may be a simple face to face or cylindrical clutch that relies upon friction. If a positive engagement is required, these faces could incorporate inter-digitations as is commonly found in “poker chip” designs that are common to the field of external fixation, as discussed above. The tool may have incorporated within it a digital readout that provides the user with discrete settings evenly spaced between the minimum and maximum angular settings of the crank arm. Accordingly, some embodiments require a separable tool to make adjustments to the connector assemblies 106′.

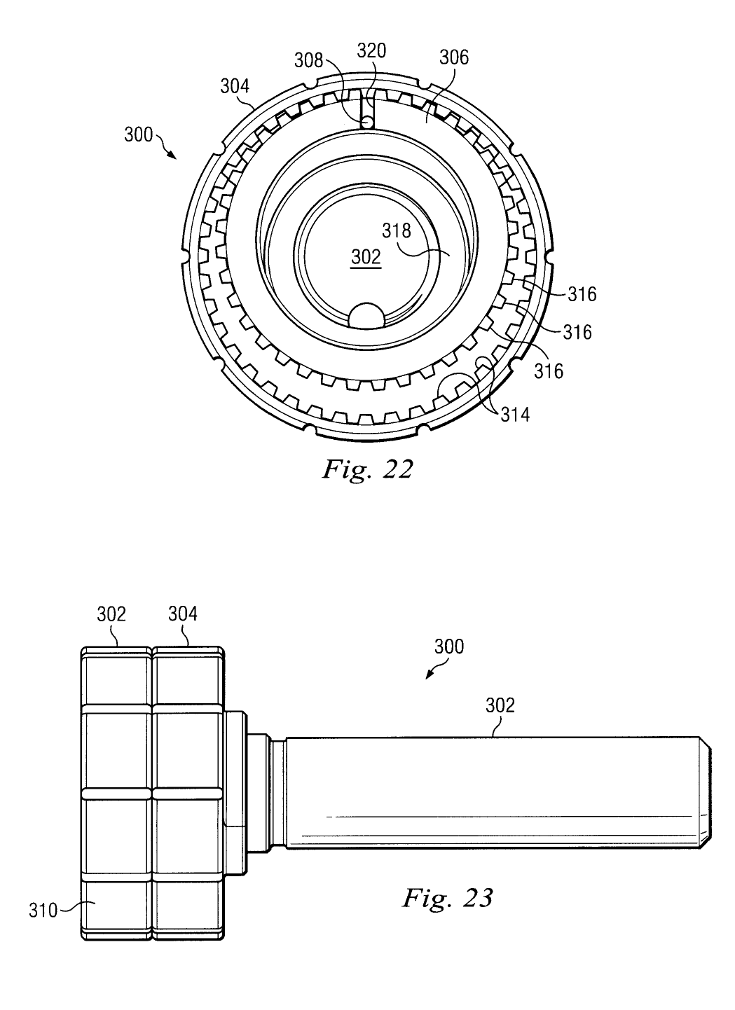

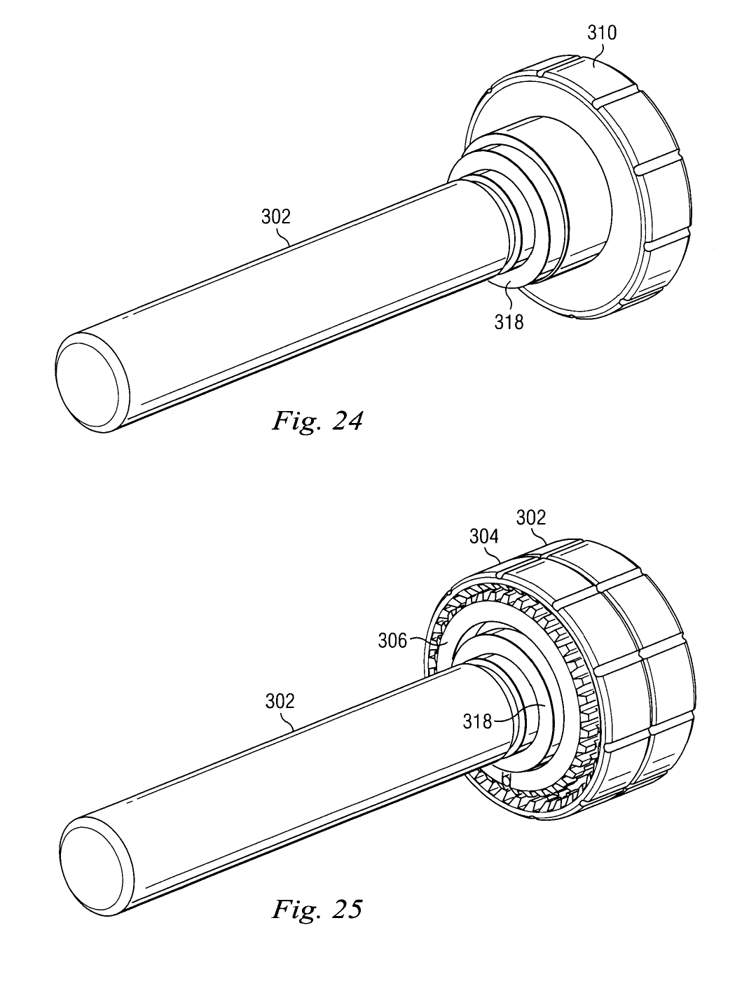

FIGS. 22-25 show the principles of another input and indicator system that may be employed in the fixation device 100. The input and indicator system is referenced herein by the numeral 300, and it employs a principle known as a hypo-cycloidal. The input and indicator system 300 includes a drive shaft 302, a dial 304, a pinion gear 306, and a fixed pin 308. The drive shaft 302 contains an input knob 310 with reference indicators such as numerical numbers printed or otherwise provided thereon about the input knob's cylindrical periphery. Coaxial with the enumerated cylindrical input knob 310 is a dial 304 with reference indicators such as numerical numbers printed or otherwise provided thereon. The dial 304 is free to turn on the same axis as the input knob 310 and its associated input shaft 302. The dial 304 includes internal gear teeth 314. In this example, the dial 304 includes forty gear teeth 314. The gear teeth 314 mesh with gear teeth 316 of the pinion gear 306. In this example, the pinion gear 306 includes 36 teeth. The pinion gear 306 is mounted to an eccentric hub 318 of the drive shaft 302 such that the teeth 314, 316 mesh. The pinion gear 306 also includes a slot 320 which accepts the fixed pin 308. When the input knob 310 is rotated by a user, the drive shaft 302 correspondingly rotates. As the drive shaft 302 rotates, the dial 304 experiences a period of dwell followed by a period of movement. In this example, with the gear teeth as described, for every one revolution of the drive shaft 302, the dial 304 dwells for 324 degrees followed by 36 degrees of rotation. The result is that the dial increments 1/10 of a turn for every revolution of the drive shaft 302, resulting in a digital readout having a range of 0 to 99. An additional stage (not shown), may be included and may be driven off the previous dial when a higher degree of resolution is required in the fixation device 100, such as when a range of 0 to 999 is desired. In the context of the fixation device 100, the drive shaft 302 replaces the drive shaft 142 shown in FIG. 6.

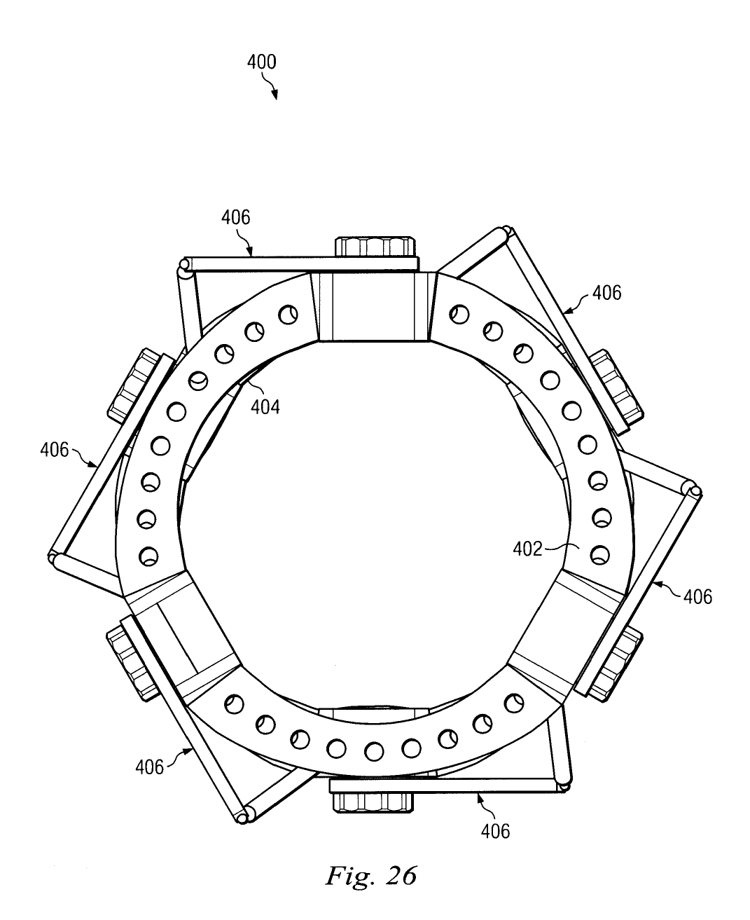

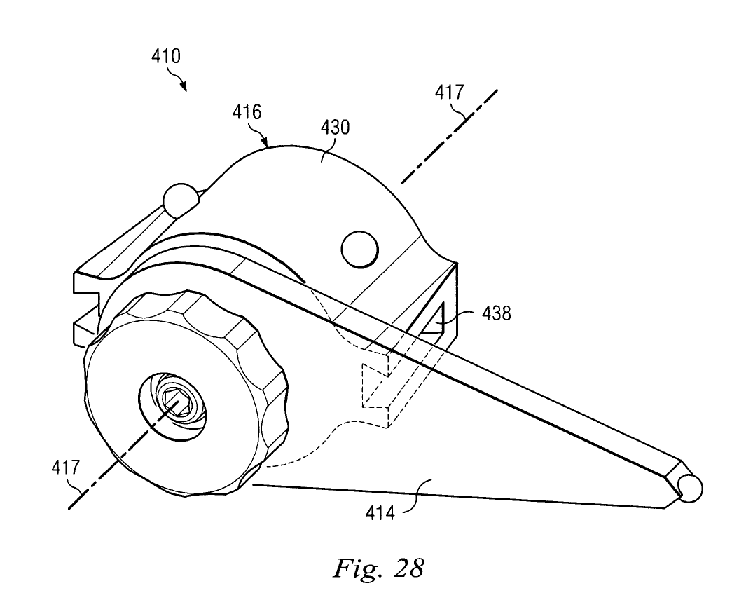

FIGS. 26-30 show yet another example of a hexapod deformity correction fixator device, referenced herein by the numeral 400. To avoid duplicity, much of the discussion above is relevant to the fixation device 400 and will not be repeated. However, the fixation device 400 differs from the above-described fixation device 100 in several respects. For example, the fixation device 400 permits rotation about a single axis by removing the cycloidal reduction mechanism that requires that the input member orbit the axis of rotation of the subsequent stage while rotating about its own axis. Accordingly, the fixation device 400 avoids the necessity of a coupling mechanism, described above as an Oldham coupler 178, that allows for radial misalignment. In addition, the fixation device 400 is arranged to provide decreased levels of radial lash and back lash (circumferential lash) because the gear mesh is not offset from the axis of rotation of the output stage gear 176. As will become apparent below, the fixation device 400 is a less complex approach using single-stage gear reduction mechanism that can be clutched and whose positional indication is inherent in the parts that make up the input, output, and ground reference members as opposed to being a secondary mechanism that acts in concert.

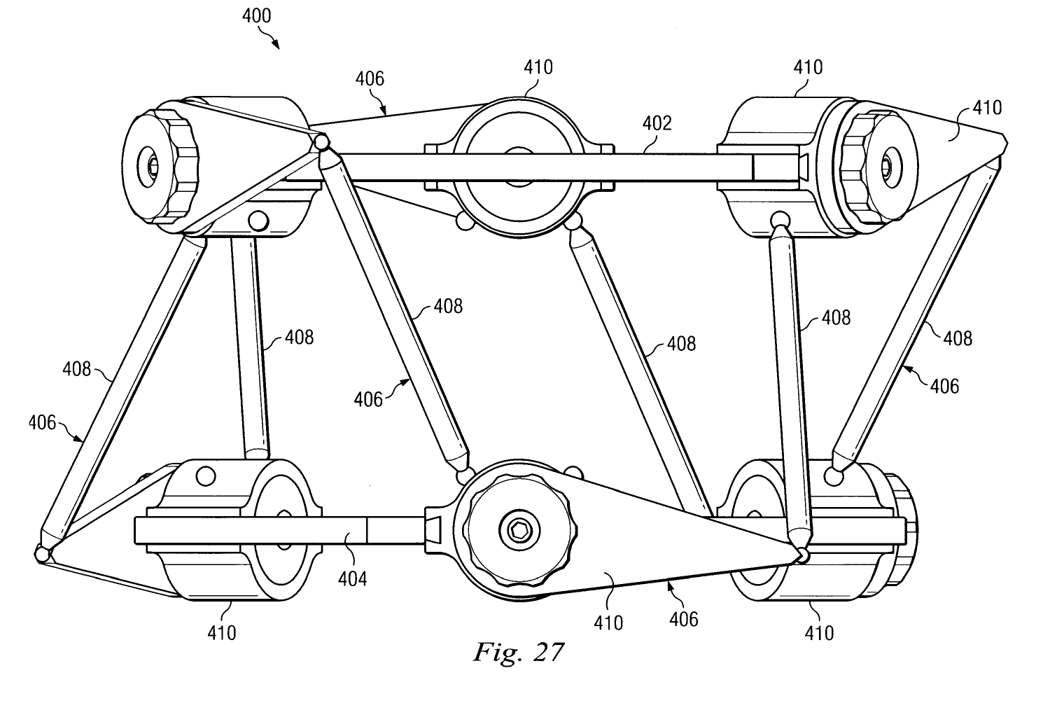

Referring to FIGS. 26 and 27, the device 400 includes a first ring element 402 and a second ring element 404, connected by six connector assemblies 406 that control the relative positioning of the ring elements 402, 404. In this example however, the ring elements 402, 404 are comprised of three ring segments connected by the connector assemblies 406. Accordingly, instead of including a ring element formed as a single, monolithic piece as in the fixation device of FIG. 1, the ring elements 402, 404 are formed of multiple segments connected to form a ring. As such, the ring elements 402, 404 may be built around a limb and need not be slid over a limb, which can be difficult in some circumstances.

The connector assemblies 406 discretely extend from and connect the first and second ring elements 402, 404. The connector assemblies 406 each include a fixed length strut 408 and a crank assembly 410 connected by a spherical joint 412. In this example, three crank assemblies 410 are associated with the first ring element 402 and three crank assemblies 410 are associated with the second ring element 404. In this example however, the connector assemblies 406 may be considered to form a part of the ring elements, as they connect the ring segments to form the ring elements. In alternative embodiments, the connector assemblies 406 are configured in paired sets much like the fixation device 100 described above.

The crank assemblies 410 in this example operate in manner similar to that discussed above in that they rotate about a fixed axis of revolution. As such, their distal end travels in an arcing direction substantially within a plane. The crank assemblies 410 include a crank arm 414, an adjustment assembly 416, and an axis of revolution 417. Additionally, as previously described these adjustment assembly 416 may be packaged into a tool that would be removed and attached as necessary to the crank arm 414 and/or the fixed length strut 408, reducing the number of components and weight of the device 400 as a whole.

The crank arm 414 includes a distal end 418, a proximal end 420, and a passage 422 at the proximal end 420. The adjustment assembly 416 includes a housing 430 and a drive assembly, an adjustment mechanism, and an input and indicator system that are combined and referred to herein as a rotating mechanism 432.

The housing 430 in this example is arranged to interface with and connect to ring segments to form a part of the ring elements as discussed above. As such, the housing 430 includes ring connection features, shown here as grooves 438. Although the embodiment shown includes the grooves 438 formed therein to receive ends of the ring segments to couple the segments, in some alternative embodiments, the housing 430 is formed integral with the first or second ring elements 402, 404 to which it is associated with and in other alternative embodiments, the housing 430 is attached to the ring using a fastening mechanism. As mentioned previously, in some embodiments, the housing 430 is configured as an individual gear reduction mechanism or may be configured in pairs.

The housing 430 in this example includes an internal bore 436 configured to receive the rotating mechanism 432. The housing 430 serves as the ground reference for the gear reduction mechanism of the rotation mechanism 432. In this example, the internal bore 436 of the housing 430 includes integral bearing races 440 and 442. Running in these races are bearing sets 444. These bearing sets 444 support the entire rotating mechanism 432 within the housing 430.

The rotating mechanism 432 includes a female spline 446, a flexspline cup 448, a standoff 450, a knob wave generator 452, and an elliptical bearing 454. An endplate 456 is fixed to the housing 430 and is arranged to interface with the flexspline cup 448 to act as a clutch that selectively engages and disengages the rotating mechanism 432. A cap screw 457 engages the standoff 450.

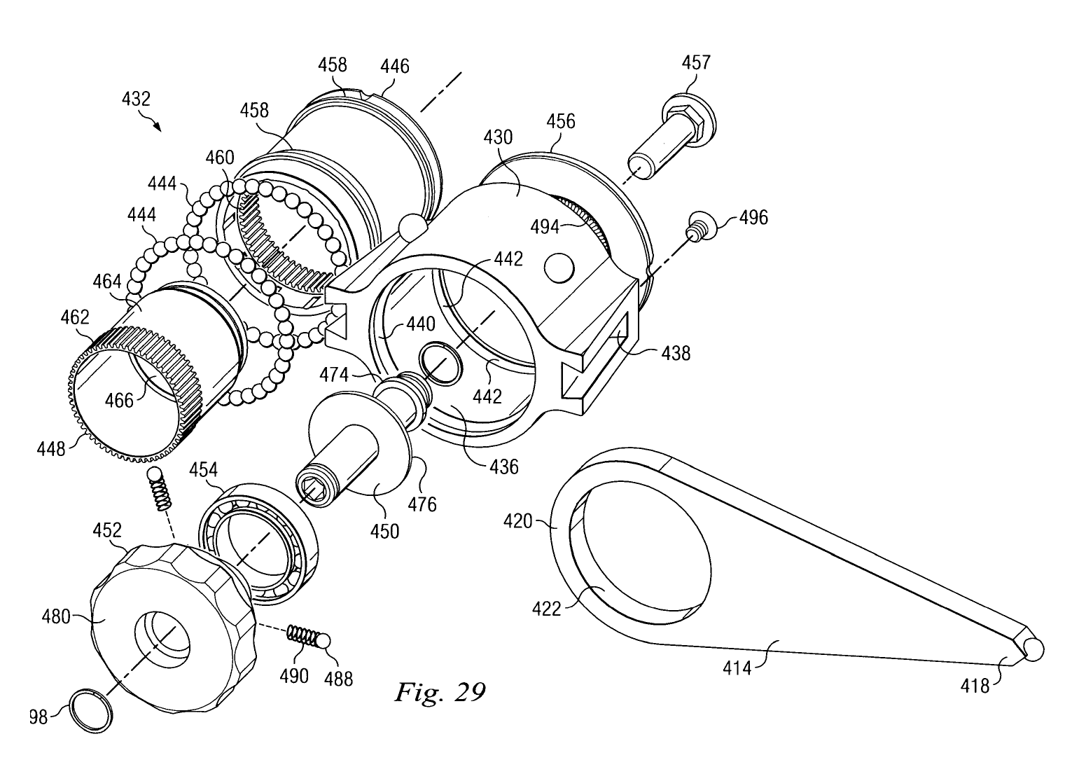

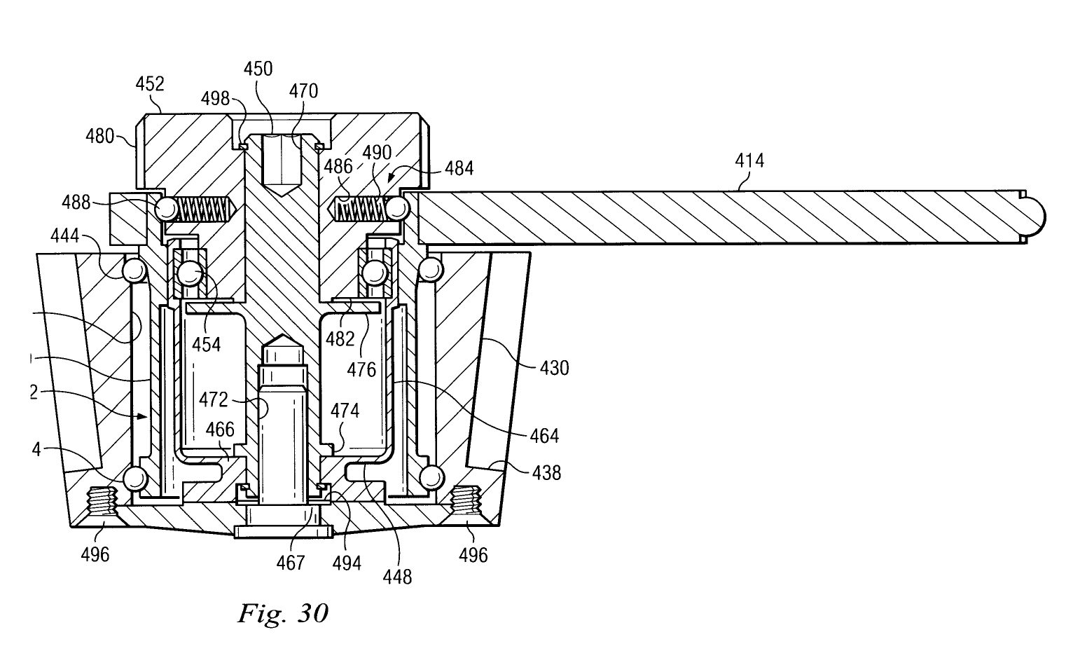

The female spline 440 is a substantially cylindrical element including an exterior surface comprising bearing races 458 disposed substantially at end regions that interface with and run on the bearing sets 444. The female spline 440 also serves as an output member that couples to the crank arm 414. As best seen in FIGS. 29 and 30, the female spline 440 contains internal teeth 460 of the output gearing that couples with external teeth 462 on the flexspline cup 448. In this example, the female spline 440 includes 180 gear teeth. Adjacent the external teeth 462, and in this embodiment adjacent one end, the female spline 440 includes a series of spaced impressions 462 that act as detents. In this example the female spline 440 includes ten impressions 462.

The flexspline cup 448 is a cup-shaped element a cylindrical portion 464 and a bottom 466. The cup 448 is shaped to fit within the female spline 440, so that the flexspline cup 448 and the female spline 440 share a common axis. As will become apparent from the discussion below, the flexspline cup 448 is configured to flex slightly to take on an elliptical shape so that external teeth 462 of the flexspline cup 448 engage the internal teeth 460 of the female spline 440 at two locations on opposing sides of the axis 417. Here, the flexspline cup 448 has a different number of teeth than the female spline 440. For example, the flexspline cup 448 includes 178 gear teeth. The bottom 466 of the cup 448 includes a plurality of radial serrations 467 that selectively engage the endplate 456.

The standoff 450 is a cylindrical post extending along the axis 417 through the flexspline cup 448. It includes a first end with a hex driving socket 470 shaped to receive a driving tool. It includes a second end with a threaded bore 472 that engages threads of the cap screw 457. It includes first and second flanges 474, 476 extending radially therefrom. When inserted into the flexspline cup 448, the first flange 474 abuts the bottom 466 of the cup-shape of the flexspline cup 448 as shown in FIG. 30.

The knob wave generator 452 is shaped to extend partially into the elliptical bearing 454 and into the open end of the flexspline cup 448. It includes a knob 480, a bearing end 482, and a detent assembly 484. The knob 480 is maintained outside the flexspline cup 448 and is arranged for grasping by a user. The bearing end 482 is elliptically shaped to match the elliptical bearing 454 and extends into the elliptical bearing 454 and into the flexspline cup 448. The bearing end 482 abuts the second flange 476 on the standoff 450. The detent assembly 484 is formed of a series of radial holes 486 formed in the knob wave generator 452. The holes 486 are located to lie adjacent the impressions 462 in the female spline 446 when the knob wave generator 452 is disposed in place. Each detent assembly 484 includes a locking ball 488 and a biasing member 490, shown as a coil spring. The biasing member 490 and the locking ball 488 are disposed within the hole 486 in manner than the biasing member 490 applies pressure outwardly on the locking ball 488 so that the locking ball 488 and the impressions 462 in the female spline 446 form a detent. Accordingly, as the knob 480 is rotated, the locking ball 488 engages and disengages with the impressions 462. This provides a tactile feedback to a user turning the knob 480. In the example shown, four detent assemblies are included in the knob wave generator. Other examples may include more or few detent assemblies.

The cap screw 457 and end plate 456 connect the rotating mechanism 432 to the housing 430. The cap screw 457 is threadably received in the threaded bore 472 of the standoff 450 and the end plate 456 connects to the housing 430. In this case, the end plate 456 is held by fasteners to the housing 430. The endplate includes radial serrations 494 that selectively engage the radial serrations 467 on the flexspline cup 448.

As will become apparent from the discussion below, the interface between the female spline 440 and the flexspline cup 448 form the gear reduction system of the fixation device 400. The gear reduction ratio may be determined based on the number of gear teeth, and may be selected to any desired reduction ratio. In this example, it is desired to have a reduction ratio of 90:1. To achieve this, the female spline 446, includes 180 internal gear teeth 460, and the flexspline cup 448 has 178 external teeth 462. The internal and external gear teeth 460, 462 have substantially identical circular/diametral pitches with pitch diameters corresponding to the number of teeth they each have, since they are both concentric to the same axis of rotation and they have a differing number of teeth and therefore different pitch diameters. The elliptical bearing 454 forces the flexspline cup 448 to flex and take on an elliptical shape causing the flexspline cup 448 to engage with the female spline 446. The major axis of this elliptical bearing 454 forces the lesser diameter of the flexspline cup 448 into engagement with the female spline 446 along the same major axis. Since the flexspline cup 448 has two fewer teeth than the female spline 446, if each successive tooth of flexspline cup 448 is forced into mesh of the next successive tooth in the female spline 446 by the action of rotation of the inner race of the elliptical bearing 454, then for each complete revolution of the elliptical bearing 454, the female spline 446 experiences a two tooth retrograde rotation relative to the flexspline cup 448. Dividing 2 into the number of teeth on the female spline 446 results in a reduction ratio of 180/2=90:1.

The flexspline cup 448 is centered on the standoff 450 and is coupled to the endcap 456 with the cap screw 457. The endcap 456 is then affixed to the housing 430 with two flat head screws 496. The endcap 456 serves as both a support for the internal workings and as a clutching mechanism. The array of serrations 494 on the inner face of the endcap 456 is configured in much the same way as those on a poker chip. These serrations mate up with a similar set of serrations 467 on the flexspline cup 448. Both the engagement and disengagement of these serrations is driven by the standoff 450. When hex 470 is turned in a clockwise fashion, the standoff threads itself onto the cap screw 457, and by the action of the flange 474 of the standoff 450 bearing on the inner surface of the flexspline cup 448 it pulls the flexspline cup 448, and the endcap 456 together causing the serrations to engage. Turning the hex 470 in a counter clockwise direction causes a snap ring 498 to bear upon the outer surface of the flexspline cup 448 pulling the flexspline cup 448 away from the endcap 456 causing the serrations to disengage. Once the serrations are disengaged the entire gear reduction mechanism is free to rotate within the housing 430 allowing for gross manual adjustment. In this embodiment the number of serrations is 90 which is equivalent to the resolution of the gear reducer. Maintaining this relationship allows that the increment of manual adjustment be equivalent to one full rotation of the gear reducer input which will be important when setting up the indication mechanism. The knob wave generator 452 rotates about the standoff 450 and has an elliptical form that forces the elliptical bearing 454 into its elliptical shape and is contained by the snap ring 498. Rotation of the knob wave generator 452 about the standoff 450 forces the inner race of the elliptical bearing 454 to follow forcing the outer race to take the form of the inner while it remains rotationally locked to the flexspline cup 448. This is the motion that forces each successive tooth into engagement with the next, resulting in the desired reduction ratio. The series of ball detent assemblies 484 are spaced evenly around the knob wave generator's circumference so that a balanced action is obtained through this multitude of detent mechanisms. This however is optional. In this assembly, the detent assemblies 484 mate up with the array of ten cylindrical impressions 462 in the female spline 446. The number ten was chosen to create ten preferential stops such that a base ten, 2-digit indication system could be created.

The indication system in this embodiment is comprised of two parts or digits providing an increment range of 00 to 99. The first of the parts or digits (not shown) are indicators provided on the housing 430 adjacent the crank arm 414. Accordingly, as the crank arm moves relative to the housing 430, its location relative to the indicators can be visually observed. In a 90:1 construct these indicators, which may be digits, would be at 4 degree intervals. The second indicator or digit is disposed between the knob wave generator 452 and the crank arm 414. Accordingly, either the indicators are imprinted on either the knob 498 or the crank arm 414. Each of the ten increments here represents a 0.4 degree increment. For simplicity of reading, the digits of the readout will follow simple base 10 numbering rather than having a fractional indication series. If desired one could either increase the number of detents or increase the reduction ratio if finer resolution was required.

It is evident that the particular illustrative embodiments disclosed above may be altered or modified and all such variations are considered within the scope and spirit of the present invention.

I claim:

1. An external fixation system comprising:
a first base member configured to attach to a first bone segment;
a second base member configured to attach to a second bone segment;
a plurality of single DOF controlling elements extending between the first and second base members, the single DOF controlling elements comprising:
a crank arm having a strut interface and a base member interface, the crank arm having a first longitudinal axis and being pivotably disposed relative to one of the first and second base members,
a strut extending from the strut interface toward one of the first and second base members, the strut having a second longitudinal axis, the crank arm and strut being structurally configured so that the first and second longitudinal axes form a non-zero angle.

2. The fixation system of claim 1, further comprising a bone engaging element configured to penetrate the first bone segment, wherein the first base member is configured to attach to the first bone segment by the bone engaging element being fixed in place relative to the first base member.

3. The fixation system of claim 1, wherein the first and second base members are complete or partial rings sized to extend about the respective first and second bone segments.

4. The fixation system of claim 1, wherein the strut connects to one of the first and second base members at a spherical articulating joint and the strut connects to the crank arm at a spherical articulating joint.

5. The fixation system of claim 1, wherein the strut has a fixed length.

6. The fixation system of claim 1, further comprising a crank assembly, the crank assembly comprising the crank arm and a gear reduction system.

7. The fixation system of claim 6, wherein the gear reduction system is separable from the crank arm.

8. The fixation system of claim 7, wherein the gear reduction system is disposed on a tool usable to rotate the crank arm.

9. The fixation system of claim 1, further comprising an input knob configured to be rotated by a user to effect movement of the crank arm.

10. The fixation system of claim 9, wherein the input knob drives a gear reduction system.

11. The fixation system of claim 10, wherein the input knob further comprising a reference indicator representing rotation of the crank arm.

12. The fixation system of claim 11, wherein the reference indicator comprises digits imprinted on the input knob.

13. The fixation system of claim 1, further comprising a crank assembly arranged so that the crank arm pivots about a longitudinal axis of revolution.

14. The fixation system of claim 13, wherein the axis of revolution projects substantially radially from the first base member.

15. The fixation system of claim 1, further comprising a crank assembly limiting the motion of the crank arm so that the strut interface on the crank arm travels substantially within a single plane.

16. The fixation system of claim 1, wherein at least one of the first and second base members comprises an edge, and the crank assembly travels in a plane substantially tangent to the edge.

17. An external fixation system comprising:
a first ring element having perimeter edges and being configured to attach to a first bone segment;
a second ring element having perimeter edges and being configured to attach to a second bone segment, the edges of the first and second ring elements defining a boundary space directly therebetween; and
a plurality of single DOF controlling elements connecting the first and second base members, the single DOF controlling elements being disposed to extend at least partially outside the boundary space directly between the first and second rings.

18. The external fixation system of claim 17, wherein the single DOF controlling elements comprise:
a crank arm having a strut interface and a ring element interface, the crank arm having a first longitudinal axis and being pivotably disposed relative to one of the first and second base members; and
a strut extending from the strut interface toward one of the first and second base members, the strut having a second longitudinal axis, the crank arm and strut being structurally configured so that the first and second longitudinal axes form a non-zero angle.

19. The external fixation system of claim 18, comprising a spherical joint disposed between the crank arm and the strut.

20. The external fixation system of claim 19, wherein the spherical joint is a first spherical joint disposed at a first end of the strut, and further comprising a second spherical joint disposed at a second end of the strut.

21. The external fixation system of claim 17, wherein the single DOF controlling elements each comprise a crank arm structurally limited to pivot about a single axis.

22. The external fixation system of claim 18, further comprising a gear reduction system associated with the crank arm.

23. The external fixation system of claim 22, wherein the gear reduction system is separable from the crank arm.

24. The external fixation system of claim 23, wherein the gear reduction system is disposed on a tool usable to rotate the crank arm.

25. The external fixation system of claim 21, wherein the single axis extends in a substantially radial direction from one of the first and second ring elements.

26. A DOF controlling element for varying the relative position of two bone attachment members, comprising:
a crank arm having a strut interface and a base member interface, the crank arm having a first longitudinal axis and being pivotably disposed relative to one of the bone attachment members; and
a strut extending from the strut interface toward one of the first and second base members, the strut having a second longitudinal axis, the crank arm and strut being structurally configured so that the first and second longitudinal axes form a non-zero angle.

27. The DOF controlling element of claim 26, comprising a crank assembly, the crank assembly limiting the motion of the strut interface of the crank arm to a single plane.

28. The DOF controlling element of claim 26, comprising an input element and a gear reduction system that reduces input motion to movement of the crank arm.

29. The DOF controlling element of claim 26, comprising a spherical joint disposed between the crank arm and the strut.

30. The DOF controlling element of claim 29, wherein the spherical joint is a first spherical joint disposed at a first end of the strut, and further comprising a second spherical joint disposed at a second end of the strut.

31. A DOF controlling element for varying the relative position of first and second bone attachment members, comprising:
a crank assembly including a crank arm having a distal end and a proximal end, the crank assembly being connectable to the first bone attachment member and being configured to permit motion of the crank arm about a single axis extending transverse to the crank arm; and
a strut having a first end and a second end, the first end being connectable to the distal end of the crank arm, the second end being connectable to the second bone attachment member.

32. An external fixation system comprising:
a first base member configured to attach to a first bone segment, the first base member having a ring-shape;
a second base member configured to attach to a second bone segment, the second base member having a ring-shape;
six single DOF controlling elements disposed at substantially equal intervals extending between the first and second base members, each of the single DOF controlling elements comprising:
a crank arm having a strut interface and a base member interface, the crank arm having a first longitudinal axis and being pivotably disposed relative to one of the first and second base members,
a strut extending from the strut interface toward one of the first and second base members, the strut having a second longitudinal axis, the crank arm and strut being structurally configured so that the first and second longitudinal axes form a non-zero angle.

33. A dial indicator system for adjusting the relative position of two bone attachment members of an external fixation device; comprising:
an input knob portion configured to receive an input from user;
a first gear portion rotatable with the input knob portion, the gear portion having gear teeth extending only partially around the gear portion;
a second gear portion engagable with the first gear portion, the second gear portion being configured to turn only when teeth of the second gear portion engage teeth of the first gear portion; and
an indicator associated with the second gear portion for viewing by the user.

34. The dial indicator system of claim 33, wherein the second gear portion includes teeth that extend only partially along its axis.
